(19) United States
(12) Patent Application Publication
(10) Pub. No.: US 2006/0171914 A1
(43) Pub. Date: Aug. 3, 2006

(54) Botanically derived cleanser

(76) Inventor: Amy M. Danles, Los Angeles, CA (US)

Correspondence Address: HARNESS, DICKEY & PIERCE, P.L.C., P.O. BOX 828, BLOOMFIELD HILLS, MI, 48303, US

(21) Appl. No.: 11/340,197

(22) Filed: Jan. 26, 2006

Related U.S. Application Data
(60) Provisional application No. 60/648,532, filed on Jan. 31, 2005.

(51) Int. Cl. A61K 8/97; C09K 3/22

(52) U.S. Cl. 424/74; 510/214

(57) ABSTRACT

A cleanser is disclosed that includes a cleansing agent derived from a botanical matter. Each cleansing agent of the cleanser is derived from botanical matter. The cleanser also includes at least one aromatic agent derived from botanical matter. Each aromatic agent of the cleanser is derived from botanical matter. The cleanser further includes a carrier adapted for carrying the cleansing agent and the aromatic agent.

### CROSS-REFERENCE TO RELATED APPLICATIONS

This application claims the benefit of U.S. Provisional Application No. 60/648,532, filed on Jan. 31, 2005. The disclosure of the above application is incorporated herein by reference.

### FIELD

The present disclosure relates to a cleanser and, more particularly, relates to a botanically derived cleanser.

### BACKGROUND

The statements in this section merely provide background information related to the present disclosure and may not constitute prior art.

Various cleansers exist for cleaning objects. For instance, disposable washcloths are commonly used to cleanse skin and other surfaces. These washcloths are particularly useful for cleansing a child's skin because the washcloths are easy to handle, they are relatively inexpensive, and because they are disposable. Cleansing salves also exist that can be spread on wounds. These salves often contain an antibacterial agent for cleaning and helping to heal the wound.

However, existing cleansers typically contain artificial chemicals. Disposable washcloths, for instance, typically contain alcohol and/or chloride, and these chemicals can irritate and dry the skin and can have an unpleasant aroma. Also, it is foreseeable that objects cleaned with the washcloth might be inserted into the mouth, especially when using the washcloth near a child. For instance, if a child's hands are cleaned with the washcloth, the child may subsequently suck on one of the cleaned fingers, and in so doing, the child may ingest harmful chemicals from the washcloth. Similarly, pacifiers, nipples for bottles, teething rings, and other objects intended for the mouth often fall on the floor or otherwise contact germs, but the child could ingest potentially harmful chemicals if these objects are cleaned with a conventional disposable washcloth. Also, cleaning objects with a conventional disposable washcloth could leave a disagreeable taste and/or smell that could upset the child.

Likewise, conventional cleansing salves typically contain artificial chemicals. These chemicals can be harmful if ingested. Furthermore, these salves can have an unpleasant smell due to the artificial chemicals contained therein.

Therefore, there is an ongoing need in the art for a cleanser that effectively cleans objects and yet is less likely to irritate, dry, or otherwise harm a person's skin. There is also an ongoing need for a cleanser that has a pleasant and soothing aroma. In addition, there is an ongoing need for a cleanser that is unlikely to leave an unpleasant taste on a cleaned object. Further, there remains a need for a cleanser that can be safely applied to objects that may be inserted into the mouth.

### SUMMARY

A cleanser is disclosed that includes a cleansing agent derived from a botanical matter. Each cleansing agent of the cleanser is derived from botanical matter. The cleanser also includes at least one aromatic agent derived from botanical matter. Each aromatic agent of the cleanser is derived from botanical matter. The cleanser further includes a carrier adapted for carrying the cleansing agent and the aromatic agent.

A method of preparing a cleanser is also disclosed. The method includes the step of providing a cleansing agent derived from botanical matter, such that each cleansing agent provided for the cleanser is derived from botanical matter. The method also includes providing at least one aromatic agent derived from botanical matter, such that each aromatic agent provided for the cleanser is derived from botanical matter. Furthermore, the method includes adding the cleansing agent and the aromatic agent to a carrier.

In addition, a cleanser is disclosed that includes between approximately two and three tablespoons of grapefruit seed extract. The cleanser also includes between approximately three and four teaspoons of chamomile oil. The cleanser also includes between approximately three and four tablespoons of lavender oil. In addition, the cleanser includes approximately one liter of water within which the grapefruit seed extract, chamomile oil, and lavender oil are dissolved.

Further areas of applicability will become apparent from the description provided herein. It should be understood that the description and specific examples are intended for purposes of illustration only and are not intended to limit the scope of the present disclosure.

### DETAILED DESCRIPTION

The following description is merely exemplary in nature and is not intended to limit the present disclosure, application, or uses.

A novel cleanser is disclosed, which includes a cleansing agent derived from botanical matter. As will be discussed, each cleansing agent of the cleanser is derived from botanical matter. The cleanser also includes at least one aromatic agent. As will be discussed, each aromatic agent of the cleanser is derived from botanical matter.

In one embodiment, the cleanser also includes a carrier adapted for carrying the cleansing agent and the aromatic agent(s). The cleansing agent and the aromatic agent(s) can be conveniently applied to objects using the carrier as will be discussed. For instance, in one embodiment, the carrier is a sheet made from wood products, and the cleansing agent and aromatic agent(s) are deposited on the sheet such that the cleanser is a cleansing washcloth. In another embodiment, the carrier is a wax within which the cleansing agent and the aromatic agent(s) are distributed such that the cleanser is a cleansing salve.

Furthermore, in one embodiment, the cleansing agent is grapefruit seed extract. As is generally known, grapefruit seed extract is prepared from the seeds, pulp, and white membranes of grapefruits. As is also generally known, grapefruit seed extract has antibacterial, antiviral, antiseptic, antifungal, and antimicrobial properties. Grapefruit seed extract is also generally nontoxic and hypoallergenic. As such, the grapefruit seed extract naturally and safely disinfects objects.

Moreover, in one embodiment, the cleanser includes a plurality of aromatic agents, including chamomile oil and lavender oil. However, the cleanser could include any suitable number and types of aromatic agents without departing from the scope of the present disclosure. Chamomile oil has a botanical name of Chamaemelon Nobile. Lavender oil has a botanical name of Lavendula Angustifolia. The chamomile oil and the lavender oil each have a pleasant aroma. These essential oils also have a pleasant taste. It will be appreciated that the chamomile oil and lavender oil provide a pleasant and soothing aroma to the cleanser and tend to mask the more pungent aroma of the grapefruit seed extract. Furthermore, because of these essential oils, objects cleaned with the cleanser are unlikely to have a disagreeable taste.

The cleanser can be dissolved within an aqueous solution. For example, the cleansing agent and the aromatic agents are dissolved in distilled water for improved purity in one embodiment.

For instance, in one embodiment, the cleanser is prepared by providing between approximately two and three tablespoons of grapefruit seed extract, between approximately three and four teaspoons of chamomile oil, and between approximately three and four tablespoons of lavender oil. The grapefruit seed extract, chamomile oil, and the lavender oil are dissolved in approximately one liter of distilled water. Then, this aqueous solution is applied to a carrier sheet made out of wood products such that the sheet is saturated with the solution. Excess solution is removed from the sheet by applying pressure to the sheet and/or by allowing excess solution to drip from the sheet. As such, the cleanser is a washcloth that can be conveniently used for cleaning a variety of objects including, but not limited to, skin, bottles, pacifiers, and the like. In one embodiment, the carrier sheet is intended to be disposed of after one use. However, the carrier sheet could be made out of a more robust material, such as cotton or other material, such that the cleanser could be used multiple times before disposal.

The carrier sheets can be an absorbent cloth sheet such as CLINIGUARD™ DRY WIPES, Item No. SCT-744999. It will be appreciated, however, that the carrier sheet could be of any suitable type. The carrier sheets can also be of any appropriate size, and in one embodiment, the carrier sheet is a three-inch (3″) by four-inch (4″) sheet. In another embodiment, the carrier sheet is a six-inch (6″) by four-inch (4″) sheet. A plurality of sheets can be saturated with the solution, folded, and stacked within a container.

In one embodiment, the aqueous solution of grapefruit seed extract, lavender oil, and chamomile oil are contained within a sprayer. The sprayer can be an aerosol-type sprayer, a pump-type sprayer, or any other suitable type. In this embodiment, the aqueous solution can be sprayed onto an object, and then the object can be wiped clean.

In one embodiment, the cleanser further includes tea tree oil. Tea tree oil has a botanical name of Melaleuca Alternifolia. The tea tree oil is effective in treating and/or preventing skin conditions, such as diaper rash. As such, the cleanser can be applied to a child's skin while changing a child's diapers, and because of the tea tree oil, the cleanser coincidentally treats and/or prevents diaper rash.

For instance, in one embodiment, the cleanser is prepared by providing between approximately one and two teaspoons of tea tree oil, between approximately two and three tablespoons of grapefruit seed extract, between approximately three and four teaspoons of chamomile oil, and between approximately three and four tablespoons of lavender oil. The tea tree oil, grapefruit seed extract, chamomile oil, and the lavender oil are dissolved in approximately one liter of distilled water. Then, this aqueous solution is applied to a carrier sheet made out of wood products such that the sheet is saturated with the solution.

In another embodiment, the cleanser is a cleansing salve. For instance, in one embodiment, the cleanser is prepared by adding between approximately two to three calendula flowers to between approximately one half cup and one cup of olive oil and heating the combination for between approximately three to four hours. Then, the oil is strained to remove the solids, and the olive oil infused with calendula oil remains. This infused oil is added to approximately two and three tablespoons of grapefruit seed extract, between approximately three and four teaspoons of chamomile oil, and between approximately three and four tablespoons of lavender oil. Next, beeswax is added as the carrier. Enough beeswax should be added to bring the cleanser to the desired consistency. As such, the cleansing salve can be used for cleansing and helping to heal wounds.

In another embodiment of the cleansing salve, almond oil is used instead of olive oil to prepare the infused oil described above. More specifically, the infused oil can be prepared by adding two to three calendula flowers to approximately one half cup and one cup of almond oil. Then, the oil is heated and strained, and the grapefruit seed extract, the chamomile oil, and the lavender oil are added in the amounts described above. Next, beeswax is added to bring the cleanser to the desired consistency.

It will be appreciated that the cleansers disclosed herein can effectively clean and disinfect surfaces using all-natural, botanical ingredients. Importantly, the cleansers are alcohol-free, chloride-free, and are otherwise free of artificial chemicals. As such, the cleansers are unlikely to dry or otherwise irritate a person's skin. Also, when used to clean a child, the cleanser has a pleasant aroma to soothe the child. Furthermore, when applied to an object, the cleansers are unlikely to leave a residue with an unpleasant taste. The objects cleaned with the cleansing washcloth can also be inserted into the mouth without introducing harmful chemicals to the person, and as such, the cleansing washcloth of the present disclosure is well suited for cleaning pacifiers, nipples for bottles, a child's hands, toys, and other objects likely to be inserted in the mouth. Likewise, the cleansing salve of the present disclosure effectively cleanses and helps heal wounds using only natural ingredients.

What is claimed is:

1. A cleanser comprising:
a cleansing agent derived from botanical matter such that each cleansing agent of the cleanser is derived from botanical matter;
at least one aromatic agent derived from botanical matter such that each aromatic agent of the cleanser is derived from botanical matter; and
a carrier adapted for carrying the cleansing agent and the at least one aromatic agent.

2. The cleanser of claim 1, wherein the cleansing agent is grapefruit seed extract.

3. The cleanser of claim 1, wherein the at least one aromatic agent is at least one of lavender oil and chamomile oil.

4. The cleanser of claim 1, further comprising at least one of tea tree oil and calendula oil.

5. The cleanser of claim 1, wherein the carrier is chosen from a group consisting of a sheet made from wood products and a wax.

6. The cleanser of claim 1, wherein the carrier is beeswax and further comprising an infused oil that is infused with an essential oil.

7. The cleanser of claim 6, wherein the infused oil is chosen from a group consisting of olive oil and almond oil.

8. The cleanser of claim 6, wherein the infused oil is infused with calendula oil.

9. The cleanser of claim 1 comprising between approximately two and three tablespoons of grapefruit seed extract, between approximately three and four teaspoons of chamomile oil, and between approximately three and four tablespoons of lavender oil, wherein the grapefruit seed extract, the chamomile oil, and the lavender oil are dissolved within approximately one liter of water, and wherein the grapefruit seed extract, chamomile oil, lavender oil, and water are carried by a sheet made from wood products.

10. The cleanser of claim 1 comprising:
approximately one quarter cup of a solution that includes between approximately two and three tablespoons of grapefruit seed extract, between approximately three and four teaspoons of chamomile oil, and between approximately three and four tablespoons of lavender oil;
between approximately one half and one cup of an oil infused with calendula oil; and
a wax that is adapted for carrying the grapefruit seed extract, the chamomile oil, the lavender oil, and the oil infused with calendula oil.

11. A method of preparing a cleanser comprising the steps of:
providing a cleansing agent derived from botanical matter, such that each cleansing agent provided for the cleanser is derived from botanical matter;
providing at least one aromatic agent derived from botanical matter, such that each aromatic agent provided for the cleanser is derived from botanical matter; and
adding the cleansing agent and the at least one aromatic agent to a carrier.

12. The method of claim 11, wherein the cleansing agent is grapefruit seed extract.

13. The method of claim 11, wherein the at least one aromatic agent is at least one of lavender oil and chamomile oil.

14. The method of claim 11, wherein the carrier is a sheet made from wood products, and further comprising the steps of:
dissolving the cleansing agent and the at least one aromatic agent in water to form an aqueous solution; and
absorbing the aqueous solution into the sheet.

15. The method of claim 11, wherein the carrier is a wax, and further comprising the steps of:
melting the wax; and
introducing to the wax the cleansing agent, the at least one aromatic agent, and an infused oil that is infused with an essential oil.

16. The method of claim 15, wherein the infused oil is at least one of olive oil infused with calendula oil and almond oil that is infused with calendula oil.

17. The method of claim 15, wherein the wax is beeswax.

18. A cleanser comprising:
between approximately two and three tablespoons of grapefruit seed extract;
between approximately three and four teaspoons of chamomile oil;
between approximately three and four tablespoons of lavender oil; and
approximately one liter of water within which the grapefruit seed extract, chamomile oil, and lavender oil are dissolved.

19. The cleanser of claim 18, further comprising a sheet made out of wood products that carries the grapefruit seed extract, the chamomile oil, the lavender oil, and the water.

20. The cleanser of claim 18, further comprising between approximately one and two teaspoons of tea tree oil.
